(19) United States
(12) Patent Application Publication
(10) Pub. No.: US 2018/0228837 A1
(43) Pub. Date: Aug. 16, 2018

(54) STABLE IODINE SOLUTIONS WITH MEDICAL APPLICATIONS

(71) Applicants: Tanya Rhodes, Largo, FL (US); Steven Harrison, Riverside, CA (US)

(72) Inventors: Tanya Rhodes, Largo, FL (US); Steven Harrison, Riverside, CA (US)

(21) Appl. No.: 15/433,053

(22) Filed: Feb. 15, 2017

(51) Int. Cl. A61K 33/18; A61K 47/10; A61K 47/02; A61K 47/42

(52) U.S. Cl. CPC A61K 33/18; A61K 47/42; A61K 47/02; A61K 47/10

(57) ABSTRACT

A liquid or gel antimicrobial solution includes a) at least 80% of total weight of a carrier liquid comprising water, alcohol or a mixture of water and alcohol; b) at least 0.001% by weight of the solution of I₂ further comprising KI and at least 0.001% by weight of CuSO₄, and c) a metallo-peptide, the liquid or gel being substantially free of sulfamic acid. The solution or dry powder format (activated on wetting) may be carried in containers, pouches, packets, fabrics, sponges or other application systems. A dry powder composition is also available.

### BACKGROUND OF THE INVENTION

### 1. Field of the Invention

The present invention relates to the field of iodine solutions, providing functional additives to iodine solutions, and stable iodine solutions that have additional functional properties having use in medical applications.

### 2. Background of the Art

Iodine solutions have been used for over a century as a disinfectant. Further advances in the performance and stability of iodine solutions are desirable.

U.S. Pat. Nos. 8,846,067 and 8,642,057 describe antimicrobial solutions and delivery systems for them use liquid antimicrobial solutions with: at least 80% of total weight of a carrier liquid comprising water, alcohol or a mixture of water and alcohol; at least 0.0001% by weight of the solution of I₂; at least 0.0001% by weight of CuSO₄; and sufficient acid in the solution to provide a pH of less than 7.0. A buffering system is also preferable in the solution, and the solution may be provided directly to wounds, burns or other skin damage as a liquid, as a spray or as a gel.

Although prior art iodine solutions can exhibit commercially useful levels of stability, it has been found that when additional functional additives are provided to these types of iodine solutions, stability (either visual or functional) tends to be significantly impaired.

### SUMMARY OF THE INVENTION

A liquid or gel antimicrobial solution includes a) at least 80% of total weight of a carrier liquid comprising water, alcohol or a mixture of water and alcohol; b) at least 0.001% by weight of the solution of I₂ further comprising KI and at least 0.001% by weight of CuSO₄, and c) a metallo-peptide, the liquid or gel being substantially free of sulfamic acid. The solution may be carried in containers, pouches, packets, fabrics, sponges or other application systems.

### DETAILED DESCRIPTION OF THE INVENTION

There is an underlying formulation that is a basis for modification with medically useful additives that have been provided in a surprisingly stable (against either or both of clarity and/or iodine activity). It was surprising that many attempted additions of common medically useful additives to the underlying iodine solutions caused relatively rapid degradation of the physical or functional properties of the underlying iodine solution. The underlying liquid or gel antimicrobial solution includes a) at least 80% of total weight of a carrier liquid comprising water, alcohol or a mixture of water and alcohol; b) at least 0.001% by weight of the solution of I₂ further comprising KI and at least 0.001% by weight of CuSO₄, and the liquid or gel being substantially free of sulfamic acid. The inventive compositions require at least medically effective amount of c) a peptide or metallo-peptide.

Substantially free of sulfamic acid is defined as less than 0.001% of sulfamic acid and preferably less than 0.0005% by weight sulfamic acid, including 0.00% by weight (zero weight) sulfamic acid. Medically effective amounts of the peptide or metallo-peptide vary with the individual materials, but a weight percent in the solution or gel of at least 0.0001% is minimally necessary, with preferential increasing amounts of at least 0.0005%, 0.001%, 0.005%, and at least 0.01% to 5% by weight being generally acceptable. Larger amounts may be used where the peptide activity is not locally damaging or ineffective.

The compositions of the invention based on the knowledge that iodine provides an optimal antimicrobial environment but is known to be cytotoxic. Attempts at inventions to create a non-cytotoxic iodine based product have been generally unacceptable and those that have been developed often may not remain stable for commercially acceptable periods of time and have a pH too low (e.g., below 4.0,) to allow for commercial application. The present is based on a stable iodine based, efficacious antimicrobial formula that remains stable (equivalent of at least 4 months, preferably at least 6 months, more preferable at least 1 year and even up to 2 years and more), has an acceptable/optimal pH for wounds (4-7, 4.5-7 or 5.0-7.0) and has a non-cytotoxic profile, including peptide ingredients to help improve the wound healing environment.

The underlying formulations for the iodine solutions in the present technology is can be represented by:

CuSO₄.H₂O+2KI>K₂SO₄+CuI+1/2I₂

Two moles of KI are needed per mole of copper sulfate. The original preparation provided by CLYRA used 0.37 mmole of KI per 0.25 mmole of copper sulfate, meaning the copper sulfate was in stoichiometric excess. Standard preparations were later adjusted to have 2 moles of KI per mole of copper sulfate. One preparation used 5% excess KI. Its biological activity is recorded below.

Alternative methods of addition were explored. Potassium iodide was added to the buffer solution both after and before pH adjustment with KOH followed by addition of the copper sulfate. The pH was adjusted to 5.8 before addition of copper sulfate. The pH drops to about 5.35 after 20 minutes. The pH after 24 hours was 5.1. Solid precipitate is usually seen at this point but not always. The normal solution was prepared followed by copper sulfate addition and a 2 hour wait until then KI was added. The pH remained stable at 5.8 prior to the addition of KI.

The formulation C4 in the table is the equivalent of the commercial formulation without sulfamic acid.

| INGREDIENT | C1 | C4 | C2 | C3 | C5 | C6 |
|---|---|---|---|---|---|---|
| Water | 93.750 | 98.750 | 93.750 | 93.750 | 93.750 | 93.750 |
| NaCl USP | 0.9000 | 0.9000 | 0.9000 | 0.9000 | 0.9000 | 0.9000 |
| CuSO₄—5(H₂O) | 0.0625 | 0.0625 | 0.0625 | 0.0625 | 0.0625 | 0.0625 |
| KI USP | 0.0625 | 0.0625 | 0.0625 | 0.0625 | 0.0625 | 0.0625 |
| Water | 6 | 0 | 2.1 | 8.1 | 7.6 | 7.1 |
| EtOH Stock | 0.000 | 0.000 | 2.9 | 0.000 | 0.000 | 0.000 |
| (Lyphazome) | | | | | | |
| EtOH | 0.000 | 0.000 | 0.1 | 0.000 | 0.000 | 0.000 |
| Thymulen 4 | 0.000 | 0.000 | 0.000 | 0.000 | 1.000 | 0.000 |
| BG | | | | | | |
| Cu Peptide | 0.000 | 0.000 | 0.500 | 0.000 | 0.000 | 0.000 |
| Na Lactate | 0.000 | 0.000 | 0.000 | 3.50 | 0.000 | 0.000 |
| Ultrapure | 0.000 | 0.000 | 0.000 | 0.000 | 0.000 | 1.500 |
| Polymer | | | | | | |
| Visual | Clear | Clear | Opaque | Clear | Clear | Opaque |
| | Lt. | Colorless | | Colorless | Colorless | White |
| | Blue | | | | | |
| pH | 6.05 | 5.15 | 5.15 | 5.22 | 5.21 | 4.57 |

Over a four month accelerated 3 to 6 months at 40° C. (90° F.) period, Examples C1. C2, C3 and C5 remained more stable than C4 and C6 with respect to both pH and visual clarity. Thymulin is a youth hormone for promoting tissue soft healing. It is a nonapeptide. These metallo-peptides are well evidenced in the field as in www.umich.edu/˜vlpecqrp/research/metallopeptide.html which is incorporated herein by references as evidencing the use of many different metal atoms as components of the peptide systems. Metals besides the common copper compounds (e.g., tin, bismuth, platinum, iron, tin, and more) are discussed with regard to at least structural and physical properties.

Copper peptide GHK-Cu is a naturally occurring copper complex of a glycyl-L-histidyl-L-lysine peptide. Since it has three amino acids it is called tripeptide. The GHK-Cu tripeptide has strong affinity for copper (II) and was first isolated from human plasma.

Copper peptides are naturally occurring small protein fragments that have high affinity to copper ions. In human plasma, the level of GHK-Cu is about 200 μg/ml at age 20. By the age of 60, the level drops to 80 μg/ml. In humans, tripeptide GHK-Cu can promote activation of wound healing, attraction of immune cells, antioxidant and anti-inflammatory effects, stimulation of collagen and glycosaminoglycan synthesis in skin fibroblasts and promotion of blood vessels growth. Recent studies revealed its ability to modulate expression of a large number of human genes, generally reversing gene expression to a healthier state. Synthetic GHK-Cu is used in cosmetics as a reparative and anti-aging ingredient.

A series of animal experiments established pronounced wound healing activity of copper peptide GHK-Cu. In the dermal wounds of rabbits GHK-Cu facilitated wound healing, causing better wound contraction, faster development of granular tissue and improved angiogenesis. It also elevated the level of antioxidant enzymes.

GHK-Cu has been found to induce a systemic enhancement of healing in rats, mice, and pigs; that is, the GHK-Cu peptide injected in one area of the body (such as the thigh muscles) improved healing at distant body areas (such as the ears). These treatments strongly increased healing parameters such as collagen production, angiogenesis, and wound closure in both wound chambers and full thickness wounds.

Biotinylated GHK-Cu was incorporated into a collagen membrane, which was used as a wound dressing. This GHK-Cu enriched material stimulated wound contraction and cell proliferation, as well as increased expression of antioxidant enzymes. The same material was tested for wound healing in diabetic rats. GHK-Cu treatment resulted in faster wound contraction and epithelization, higher level of glutathione and ascorbic acid, increased synthesis of collagen, and activation of fibroblasts and mast cells. Ischemic open wounds in rats treated with GHK-copper healed faster and had decreased concentration of metalloproteinases 2 and 9 as well as of TNF-beta (a major inflammatory cytokine) compared with vehicle alone or with untreated wounds.

Copper peptide GHK-Cu is widely used in anti-aging (INCI name: Copper tripeptide-1).

Although tinylated peptides also are believed to be functional in the present technology and invention, because of copper's unique and specific biological functionality, the use of copper peptides is believed to be unique even within this narrow subgeneric group of metlalo-peptides.

Copper is a transition metal that is vital for all eukaryotic organisms from microbes to humans. A dozen enzymes (cuproenzymes) use changes in copper oxidation state to catalyze important biochemical reactions including cellular resporation), antioxidant defense (ceruloplasmin, superoxide dismutase (SOD), detoxification (metallothioneins), blood clotting (blood clotting factors V and VIII), melanin production (tyrosinase) and the connective tissue formation (lysyl peroxidase). Copper is required for iron metabolism, oxygenation, neurotransmission, embryonic development and many other essential biological processes. Another function of copper is signaling—for example, stem cells require a certain level of copper in the media to start their differentiation into cells needed for repair. Thus, GHK-Cu's ability to bind copper and to modulate its tissue level is a key factor determining its biological activity.

Peptides are divided into several classes, depending on how they are produced:

Milk Peptides

Two naturally occurring milk peptides are formed from the milk protein casein when digestive enzymes break this down; they can also arise from the proteinases formed by lactobacilli during the fermentation of milk.

Ribosomal Peptides

Ribosomal peptides are synthesized by translation of MRNA. They are often subjected to proteolysis to generate the mature form. These function, typically in higher organisms, as hormones and signaling molecules. Some organisms produce peptides as antibiotics, such as microcins. Since they are translated, the amino acid residues involved are restricted to those utilized by the ribosome.

However, these peptides frequently have post-translational modifications, such as phosphorylation, hydroxylation, sulfonation, palmitoylation, glycosylation and disulfide formation. In general, they are linear, although lariat structures have been observed.

Nonribosomal Peptides

Nonribosomal peptides are assembled by enzymes that are specific to each peptide, rather than by the ribosome. The most common non-ribosomal peptide is glutathione, which is a component of the antioxidant defenses of most aerobic organisms. Other nonribosomal peptides are most common in unicellular organisms, plants and fungi and are synthesized by modular enzyme complexes called nonribosomal peptide synthetases.

These complexes are often laid out in a similar fashion, and they can contain many different modules to perform a diverse set of chemical manipulations on the developing product. These peptides are often cyclic and can have highly complex cyclic structures, although linear nonribosomal peptides are also common. Since the system is closely related to the machinery for building fatty acids and polyketides, hybrid compounds are often found. The presence of oxazoles or thiazoles often indicates that the compound was synthesized in this fashion.

Peptides are divided into several classes, depending on how they are produced:

Milk Peptides

Two naturally occurring milk peptides are formed from the milk protein casein when digestive enzymes break this down; they can also arise from the proteinases formed by lactobacilli during the fermentation of milk.

Ribosomal Peptides

Ribosomal peptides are synthesized by translation of MRNA. They are often subjected to proteolysis to generate the mature form. These function, typically in higher organisms, as hormones and signaling molecules. Some organisms produce peptides as antibiotics, such as microcins. Since they are translated, the amino acid residues involved are restricted to those utilized by the ribosome.

However, these peptides frequently have post-translational modifications, such as phosphorylation, hydroxylation, sulfonation, palmitoylation, glycosylation and disulfide formation. In general, they are linear, although lariat structures have been observed.

Nonribosomal Peptides

Nonribosomal peptides are assembled by enzymes that are specific to each peptide, rather than by the ribosome. The most common non-ribosomal peptide is glutathione, which is a component of the antioxidant defenses of most aerobic organisms. Other nonribosomal peptides are most common in unicellular organisms, plants and fungi and are synthesized by modular enzyme complexes called nonribosomal peptide synthetases.

These complexes are often laid out in a similar fashion, and they can contain many different modules to perform a diverse set of chemical manipulations on the developing product. These peptides are often cyclic and can have highly complex cyclic structures, although linear nonribosomal peptides are also common. Since the system is closely related to the machinery for building fatty acids and polyketides, hybrid compounds are often found. The presence of oxazoles or thiazoles often indicates that the compound was synthesized in this fashion. Both natural and synthetic peptides may be used in the present technology.

Antimicrobial peptides such as within the Magainin family, Cecropin family, Cathelicidin family, and Defensin family

Tachykinin Peptides

Substance P, Kassinin, Meurokinin A, Neurokinin B, and Eledoisin.

Vasoactive Intestinal Peptides

(Vasoactive Intestinal Peptide; PHM27),

Pituitary Adenylate Cyclase Activating Peptide, (Peptide Histidine Isoleucine 27), (Growth Hormone Releasing Hormone 1-24), Glucagon and Secretin.

Pancreatic Polypeptide-Related Peptides

(NeuroPeptide Y), (Peptide YY),

(Avian Pancreatic Polypeptide), Pancreatic PolYpeptide

Opioid Peptides

Propiomelanocortin peptides, Enkephalin pentapeptides, Prodynorphin peptides, Calcitonin peptides

Calcitonin, Amylin and AGG01.

Other Peptides

B-type Natruretic Peptide (BNP) is produced in myocardium and useful in medical diagnosis, and Lactotripeptides might reduce blood pressure although the evidence is mixed.

Underlying Technology Statement

Summary Information for Patent:

The development of the present invention should be appreciated to comprehend the significance of the advance established herein. Iodine solutions have been used for many years as an antimicrobial solution. It is desired to be able to provide a composition based on KI and Cu₂SO₄ that generates sufficient I₂ (e,g., in the amount of at least 100 ppm, at least 150 ppm, at least 200 ppm and even 250 ppm to be an effective antimicrobial with commercial standards of stability. Previous work has not been able to provide a stable formula beyond 80 ppm.

Using certain solutions such as those of U.S. Pat. Nos. 8,846,067 and 8,642,057 (with sulfamic acid) formula at 250 ppm resulted in some early precipitation or discoloration, and depending on additional ingredients exhibited an undesirably low pH as well as some cytotoxicity in its profile.

To be a commercially viable proposition for the medical, veterinary and dental industry, high quality compositions should have the following attributes:
Effective antimicrobial activity against a range of common pathogens
Effective activity for at least 24 hours (3-7 days preferred)
Non-cytotoxic profile to ensure minimal detriment to healing. (Most anti-microbials such as iodine tincture, chlorhexidine, silver etc. can have a cytotoxic effect on cells).
Acceptable pH to ensure no pain or stinging on application plus no acidic/alkaline negative clinical outcomes (pH 4-7 preferred).

This work based on simple KI/Copper sulfate in water (Clyra 4). Nothing else.

The products of the present invention have been able to meet all the above criteria, and also have had the added benefit of odor control.

The underlying Clyra™ 4 composition was used as a base-line composition to is which the inventors added ingredients in an attempt to provide added unique and specific wound care benefit.

| INGREDIENT | Clyra ™ 1 (A) | Clyra ™ 4 |
|---|---|---|
| Water | 98.9340 | 98.9750 |
| Sulfamic Acid | 0.0260 | 0.0000 |
| K Bicarbonate USP | 0.0150 | 0.0000 |
| NaCl USP | 0.9000 | 0.9000 |
| CuSO₄5—H₂O USP | 0.0625 | 0.0625 |
| KI USP | 0.0625 | 0.0625 |
| | 100.0000 | 100.0000 |

Clyra™ 4 was used as the base-line control formula for stability (appearance and pH as well as antimicrobial efficacy testing) and prescription 0.12% Chlorhexidine Gluconate was used as the positive control formula for all antimicrobial efficacy testing. (as this is the standard used in dental practice commercially). A log 4 kill is required to pass based on FDA requirements. Therefore the baseline formula (Clyra™ 4) and the other formulas with peptides needed to pass a log 4 kill minimum and preferably be equivalent to the prescription chlorhexidine gluconate sample.

Many formulas, with many different additives individually associated with some level of medical benefit were tested. With the exception of the technology of the peptides of the present invention, essentially all tested materials proved unstable due to precipitation or extremely low pH. The uniqueness of the peptides with respect to enabling maintenance of high iodine concentrations and providing acceptable length of stability without precipitation is highly indicative of is unexpected results evidencing invention.

Final formulas contained peptides as wound healing additives.
LN231-199—Clyra™ 4 with Copper Peptide as a wound healing/anti-inflammatory additive
LN231-200—Clyra™ 4 with Thymulen™ 4BG100 (peptide) as a peptide for healing (youth hormone)

All passed cytotoxicity and have effective kill time at 3 days (preliminary data log 7 kill. Final Report >Log 5 kill. Both peptide formulas provided a unique medical material that is antimicrobial, stable and provides wound healing benefits.

The base formula (with or without peptides) provides a unique medical material that is antimicrobial and stable ready to be incorporated into the following formats:—
Washes
Gels
Hydrocolloids
Alginates
Foams (open or closed cell)
Superabsorbent polymers
Film formers
Creams and emulsions
Biological Cellular and acellular membranes
Gelatin capsules specifically for application to insert between gum and tooth or other areas as an antimicrobial treatment for periodontal disease. May be is a hydrolyzed gelatin cross linked, as with gluteraldehyde and/or other ingredients.

Each reference cited in this disclosure are incorporated by reference in their entirety.

As in the above cited U.S. Pat. No. 8,846,087, the solutions, gels and compositions described herein are also useful in direct medical treatment of wounds, sores, topical conditions and transdermally accessible conditions. The use of gels, solutions and compositions may be directly applied to the region of the patient (both human and non-human) where treatment is desired. As the primary ingredients (K, I, Cu and S.sub.4) are biocompatible and are generally regarded as safe (GRAS) under FDA guidelines. The active components are so safe for human consumption they appear in over-the-counter and/or prescription medication presently in the marketplace. By selection of conventional carriers commonly used or developed in the future for delivery onto skin and into wounds, in combination with the solution and ingredients used in the present technology, assists in providing an effective medicinal composition.

In addition to the use of materials described above in forming the solutions, compositions and gels (especially in the absence of the solid carriers such as fabric, sheets and layers), such as ointment bases, cream bases, emollients, dimethylfulfoxide, alginates, natural and synthetic gums (agar-agar, polysiloxanes, polymeric carriers, solvents, biocompatible carriers, and the like. These direct addition compositions and solutions may be carried on a substrate or fabric, and (as incidentally occurring in the carried compositions of U.S. Published Patent Application Document No. 20120087965), but possibly in greater concentration where the composition or solution may flow out of the carrier/fabric and directly is onto the skin or into the wound.

A carrying composition may comprises the active iodine-releasing, iodine-providing technology described herein which is effective in promoting antimicrobial activity here applied. Preferably said active ingredients comprise from about 0.01% to about 40% (including the liquid or gel carrier) by weight of the total carrier. The weight proportion of more preferably from about 0.05% to about 25%, and most preferably from about 1.0% to about 10.0% by weight may be used.

A liquid binder according to the invention is used in particular for dispersing the components, as explained below, and for enhancing the stability of the composition. Moreover, the liquid binder is used to adjust the concentrations of the active ingredients of the composition according to the invention. Obviously, the liquid binder has also additional properties, e.g. thickening properties, stabilizing properties, water-binding promoting properties as is well known to the person skilled in the art. These liquid binders are preferably selected from the liquid polyols, polymeric binders, fumed silica and gums or a combination thereof. Examples of suitable liquid polyols include glycerol, propylene glycol, polyethylene glycol (PEG). Examples of suitable gums include natural gums and modified (semi-synthetic) gums, for example acacia gum, gum arabic, caraya gum, gum tragacanth, xanthan gum and cellulose gum. Examples of suitable polymeric binders are polyvinyl pyrrolidone, casein or salts thereof, wherein the salts comprise a metal of Group 1 or Group 2 of the Periodic System. According to the invention, it is preferred that the liquid binder is glycerol, glycol, propylene glycol, PEG, fumed silica, a gum, or a combination thereof. In a particularly preferred embodiment of the present invention the liquid binder is a combination of a liquid polyol and a fumed silica, most preferably PEG 1500 in combination with fumed is silica, in total amounts of 0.005 to 4% and 1 to 20%, respectively, based on the total weight of the composition. Most preferred is an amount of PEG 1500 from about 0.01% to about 2% by weight of the total composition, and an amount of fumed silica from about 3% to about 10% by weight of the total composition. Moreover, component (a) is preferably employed as an aqueous solution comprising the binder, said aqueous solution comprising 25-75% by weight, preferably 35-65% by weight of the binder, calculated on the basis of the total weight of the aqueous solution.

Additionally, according to the invention the pH of the composition is essential for a controlled and long lasting release of the active component, i.e. oxygen. Tests have revealed that the pH is preferably in the range of 3.5-6.9, preferably 4.0-6.5 and most preferably 4.5-6.2.

The composition according to the present invention, which is used for the treatment of open wounds and even burns (to prevent protection), may further comprise a gelatinous thickener. Typically a cellulose material, such as cellulose, sodium carboxymethylcellulose, (hydroxy)propylcellulose, methylcellulose, or ethylcellulose, is used as a thickener. Preferably sodium carboxymethylcellulose is used in the present invention, in an amount of 0.2 to 4.0 percent by weight, preferably 0.5 to 2.5 percent by weight, calculated on the total weight of the composition.

The composition may further comprise an agent that counteracts loss of moisture, and that optionally also has an anti-microbial action. Preferably a carbohydrate, more preferably an alditol, such as, for example, erythritol, arabinitol, xylitol, galacitol, sorbitol, iditol, mannitol, hepitol, or octitol, is used as the agent that counteracts the loss of moisture. In the present invention the use of alditol is preferred, typically in an amount of 0.5 to 10.0 percent by weight preferably in an is amount of 1.0 to 5.0 percent by weight, calculated on the total weight of the composition.

The compositions may also contain an anti-oxidant. Examples of suitable anti-oxidants are Lipochroman-6, sodium ascorbylphosphate, or combinations thereof. Preferably the compositions contain an amount of anti-oxidant of about 0.10% to about 4.0% by weight of the total composition. In a preferred embodiment Lipochroman-6 and sodium ascorbylphosphate are used. Preferably the compositions contain from about 0.01% to about 1.0% by weight of Lipochroman-6, and from about 0.10% to about 3.00% by weight of sodium ascorbylphosphate.

The compositions and medicaments according to the present invention may additionally comprise a components selected from the group of antibiotics such as natural or synthetic antibiotics such as sulfa drugs that are used to treat bacterial and some fungal infections. Suitable sulfa drugs comprise prontosil, sulfadiazine, sulfamethizole (Thiosulfil Forte®), sulfainethoxazole (Gantanol®), sulfasalazine (Azulfidine®), sulfisoxazole (Gantrisin®), and various high-strength combinations of three sulfonamides. Preferably, the sulfa drug is sulfadiazine.

The compositions and medicaments according to the present invention my further comprise a zinc component which are beneficial in wound healing. A suitable example is zinc gluconate.

The compositions and medicaments according to the present invention further preferably comprise an agent that promotes degradation of biofilms on open wounds. Suitable agents include peroxide forming enzymes such as lactoperoxidase as is disclosed in WO 88/02600 of Poulson, incorporated by reference, and glycoproteins such as lactoferrin as disclosed in EP A 1.545.587, incorporated by reference.

The compositions according to the present invention can optionally further is comprise any pharmaceutically acceptable excipient, such as, for example, colorants, (de)odorants, preservatives and the like. The composition, according to the present invention, is intended for use in the treatment of open wounds and burns. The term “open wound”, as used herein, may refer to any type of tissue injury, but particularly to tissue injuries characterized by delay or complete failure of healing. Typical but non-limiting examples of such injuries are traumatic injury, including burns, injury resulting from surgery, diabetic wounds, pressure ulcers, arterial ulcers, decubitus ulcers, and venous stasis ulcers. The greatest benefits are achieved in injured tissues with compromised blood flow and oxygen supply.

The treatment of open wounds and burns according to the present invention typically comprises topical administration of the medicament or of a combination of the medicaments, containing the composition, to the open wound or burn. The medicament is preferably applied to the wounds or burns in amounts sufficient to completely cover the entire surface of the wound. In a preferred embodiment, the composition is applied to the open wound or burn, 1 to 8 times daily, more preferably 2 to 4 times daily. The treatment is continued as long as necessary to completely heal the wounds, it is applied to, or as long as beneficial effects are observed.

This technology includes a solid delivery system of components that forms the solution of claim 1 when contacted with water or alcohol comprising two distinct particulates, a first particulate comprising a solid particle of KI and a second particulate comprising a solid particle of CuSO₄, the two distinct particulates being present in a total combination with a metallo-peptide or L-pyroglutamyl-L-alanyl-L-lysyl-L-seryl-L-glutaminyl-glycyl-glycyl-L-seryl-L-asparagine, and then total combination being substantially free of sulfamic acid. At least one of the two distinct particulates may be independently coated with a soluble/dispersible coating is and the two reactants kept apart by the coating to avoid premature release of iodine. The solid delivery system may include compositions where the soluble/dispersible coating comprises particles of a superabsorbent polymer.

The term “substantially free of sulfamic acid” means that there is insufficient concentration of sulfamic acid to reduce the pH of deionized water at 20 C by more than 0.1 pH units (e.g., to less than or equal to 6.0). In absolute terms, this would definitely be less than 0.1N sulfamic acid, or less than 0.05N sulfamic acid. The liquid composition may also be described as having less than 0.0001% by weight of sulfamic acid, independent of any carrying media (e.g., solids and/or fabrics).

There are numerous specific embodiments in which the present technology can be embodied. One example of a number of specific devices can be found in the dental field. In that field, bot immediate, short term and longer term antimicrobial activity can be desired. One method of application of the solutions of the present invention is in carrier materials, which can be orally positioned within the mouth, on teeth or between teeth. The carrier may fabric, film, solution, or any solid carrier that allows the solution to diffuse at a needed rate. The solution may slowly diffuse from a stable solid carrier, the carrier itself may dissolve as the controlling mechanism for a release rate for the solution, a gel or gum may release the solution from a dispersion or emulsion within the gel, or thickened solution may be applied to oral or dental surfaces. For example, a pad, thick solution or gel may be placed over small wounds or against devices to be implanted during oral surgery. More permanent or intermediate applications can be done with more stable carriers such as slowly dissolvable or non-dissolvable films or solids (e.g., chips) which can be positioned for intermediate or longer terms. Polymers which allow the solutions to diffuse or which open up when in contact with moisture or dissolve, disperse or decompose may be used as carriers, alone or in combination with fabric or carrier is layers. Such films or chips may be placed between teeth or between teeth and appliances. The solutions may be used on carrier trays as solution or in tray covers so that tools may be placed on the covered trays to maintain reduced microbial growth or transmission.

Polymers may include, by way of non-limiting examples, acrylic polymers, vinyl polymers or copolymers (e.g., polyvinyl alcohol, polyvinylidence chloride, polyvinypyrrolidone, polyvinylacetate), polyethylene or polypropylene, polyamides, polysiloxanes, cellulosic polymers (natural and synthetic such as cellulose acteate), and other polymers which can carry the solutions and deliver them at a desired rate.

Although the aforementioned method of treatment generally applies, it is within the skill and within the objective of any professional, trained in the art of wound healing, to adjust the preferred amounts of the medicament and/or the frequency it is applied with, as well as the duration of the treatment, in order to optimize the efficacy for each individual patient. The materials and compositions of the invention may be applied to wounds, burns, cuts, mucosal tissue, mucosal membrane, and/or the skin for any after event medical condition or pre-event medical condition.

What is claimed:

1. A liquid or gel antimicrobial solution comprising: at least 80% of total weight of a carrier liquid comprising water, alcohol or a mixture of water and alcohol; at least 0.001% by weight of the solution of I₂ further comprising KI and at least 0.005% by weight of CuSO₄, a metallo-peptide or L-pyroglutamyl-L-alanyl-L-lysyl-L-seryl-L-glutaminyl-glycyl-glycyl-L-seryl-L-asparagine, and being substantially free of sulfamic acid.

2. The solution of claim 1 comprising at least 0.05% by weight of the metallo-peptide.

3. The solution of claim 1 comprising a liquid composition with less than 0.0001% by weight of sulfamic acid.

4. The solution of claim 2 comprising at least 0.05% by weight of the metallo-peptide.

5. The solution of claim 1 comprising at least 0.05% by weight of a copper-peptide.

6. The solution of claim 3 comprising at least 0.05% by weight of a copper-peptide.

7. The solution of claim 5 wherein the copper-peptide comprises from 1 to 9 peptide units.

7. The solution of claim 6 wherein the copper-peptide comprises from 1 to 9 peptide units.

8. The solution of claim 1 further comprising a buffering agent.

9. The solution of claim 1 with substantially no buffering agent.

10. The solution of claim 8 wherein the buffering agent comprises a carbonate, bicarbonate or phosphate.

11. The solution of claim 1 comprising at least 0.05% by weight L-pyroglutamyl-L-alanyl-L-lysyl-L-seryl-L-glutaminyl-glycyl-glycyl-L-seryl-L-asparagine.

12. A solid delivery system of components that forms the solution of claim 1 when contacted with water or alcohol comprising two distinct particulates, a first particulate comprising a solid particle of KI and a second particulate comprising a solid particle of CuSO₄, the two distinct particulates being present in a total combination with a metallo-peptide or L-pyroglutamyl-L-alanyl-L-lysyl-L-seryl-L-glutaminyl-glycyl-glycyl-L-seryl-L-asparagine, and then total combination being substantially free of sulfamic acid.

13. The solid delivery system of claim 12 wherein at least one of the two distinct particulates is independently coated with a soluble/dispersible coating and the two reactants kept apart by the coating to avoid premature release of iodine.

14. The solid delivery system of claim 13 wherein the soluble/dispersible coating comprises particles of a superabsorbent polymer.

15. The solid delivery system of claim 13 wherein the solution comprises at least 0.05% by weight of a copper-peptide.

16. The solid delivery system of claim 13 wherein the solution comprises at least 0.05% by weight of a copper-peptide.

17. The solid delivery system of claim 12 wherein the copper-peptide comprises from 1 to 9 peptide units.

18. The solid delivery system of claim 12 wherein the copper-peptide comprises from 1 to 9 peptide units.
